(19) United States
(12) Patent Application Publication
(10) Pub. No.: US 2004/0151706 A1
(43) Pub. Date: Aug. 5, 2004

(54) Collection and storage of biological specimens containing stem cells from healthy individuals for future use in treatment of their own cytopathological illnesses or other medical conditions

(76) Inventors: Alex Shakhov, Frederic, MD (US); Alex Kogon, Hagerstown, MD (US)

Correspondence Address: ROSENBERG, KLEIN & LEE, 3458 ELLICOTT CENTER DRIVE-SUITE 101, ELLICOTT CITY, MD, 21043, US

(21) Appl. No.: 10/763,197

(22) Filed: Jan. 26, 2004

Related U.S. Application Data
(60) Provisional application No. 60/442,506, filed on Jan. 27, 2003.

(51) Int. Cl. A61K 45/00

(52) U.S. Cl. 424/93.7

(57) ABSTRACT

Method for treating a cytopathological disease or other medical condition in a mammal, including the steps of harvesting a biological specimen containing stem cells selected from peripheral blood and/or bone marrow from the body of a donor, then storing the harvested bone marrow for a predetermined waiting period before re-infusing it back into the same donor after the donor later contracts and is diagnosed with one or more of a cytopthalogical illness, a chronic fatigue syndrome, and/or damaged tissue. Then, at least a portion of the stored biological specimen containing stem cells is reintroduced in the donor, where the reintroduction occurs either after any cytotoxic therapy is performed on the donor that damages native bone marrow, or as part of another treatment used to restore or rejuvenate healthy tissue, in the donor.

### CROSS-REFERENCE TO RELATED APPLICATIONS

This application claims the benefit of priority under 35 U.S.C. ′119(e) to U.S. Provisional application serial No. 60/442,506, filed Jan. 27, 2003, the entire disclosure and contents of which are incorporated herein by reference for all purposes.

### BACKGROUND OF THE INVENTION

1. Field of the Invention

This invention relates to treatment of cytopathological diseases and other medical disorders and conditions by inoculating patients with their own healthy stem cells, which is collected in advance when the donor is healthy and stored until a medical need arises for its therapeutic re-introduction into the donor. More specifically, an autologous transplantation method is provided to rescue and restore tissue damage, tissue deterioration, and/or fatigue caused by a disease or disorder itself or an adverse side effect caused by an interventive treatment applied to the disease, in which healthier, more enriched biological specimens of stem cells are collected in advance from the body of the donor while still healthy for long term storage before reinfusion into the same donor in a post-diagnosis time frame.

2. Description of Related Art

Critical cytopathological illnesses, such as cancer, AIDS, and like, have a major economic impact on health care providers, individual patients and society. Current therapies used for these serious illnesses are highly expensive, require high technology, yet generally deliver uncertain outcomes. In view of the high and escalating financial burdens associated with treatment of such serious illnesses, recent healthcare reforms are emphasizing evidence-based healthcare and economic evaluations of therapies to assess treatments from efficacy and cost standpoints.

For instance, very high dose cytotoxic therapy (chemo- and/or radiotherapy) has been increasingly used as the most common treatment for hematological and solid malignancies. There also have been reports on using this therapy against HIV. The rationale of this approach is to overcome the chemotherapy or radiotherapy resistance of tumor cells by administering doses of cytotoxic agents at the levels terminal and “supra-terminal” both for malignant cells and cells of the patient's hematological system. However, high-dose chemotherapy or radiotherapy destroys not only cancer cells, but also destroys much of the bone marrow and stem cells. This leaves the patient vulnerable to bleeding and infection.

Bone marrow is spongy material found inside bones that contains many cell types, including stroma, vascular cells, adipocytes, osteoblasts and osteoclasts, as well as mesenchymal stem cells and hematopoietic stem cells. The stem cells produce red blood cells, white blood cells, platelets, and other components important for fighting infection, carrying oxygen and helping to control bleeding. These stem cells that transplant patients need to make new healthy marrow usually are present in bone marrow, but also are released in small numbers into the peripheral (circulating) blood. Bone marrow collection usually requires a patient to go to the operating room, receive general anesthesia, and have bone marrow withdrawn from the hip bone with a needle or syringe. Bone marrow transplantation (BMT) is a procedure in which the extracted bone marrow is stored until transplanted back into the same donor or other suitably matched different recipient. Peripheral blood stem cell transplantation (PBSCT) involves the removal of stem cells from the circulating blood by machine; a procedure referred to as apheresis.

BMT has been used in prior therapeutic efforts to reconstitute and rescue damaged and destroyed bone marrow resulting from such supra-terminal cytotoxic treatments, and to restore normal hematopoietic and immunlogical function. Stem cell transplants via BMT has primarily been used in the treatment of cancers, leukemias, anemias, and certain immune diseases. The major indications are hematological malignancies such as acute myeloid leukemia (AML), acute lymphoblastic leukemia (ALL), chronic myeloid leukemia (CML) and myelodysplastic syndromes, and in the instance of solid tumors, breast-, testicular-, small cell lung-, and/or ovarian cancers.

Restoration via BMT is typically attempted by inoculation with stem cells from three potential sources: 1) HLA-matched unrelated donors, 2) HLA-identical siblings, or 3) autologous BMT. In autologous BMT, patient's receive their own stem cells. In allogenic transplants, patients receive stem cells from someone other than themselves who is HLA matched with the recipient. Each of these sources has advantages and disadvantages, but none of them as currently practiced is completely effective and reliable.

The unrelated bone marrow for allogenic transplants is readily available, and it has been possible to store marrow for long periods of time via cryopreservation after extraction from donors. Unfortunately, even the carefully selected, human leucocyte antigen (HLA)-compatible donors present enough immunological incompatibility to necessitate the suppression of immune systems of both the donor, before the collection of bone marrow specimen, and the recipient, both before and after the transplantation, for the rest of patient's life. Therefore, allogenic transplantation is not the ideal, nor optimal treatment modality.

HLA-identical siblings demonstrate much higher level of compatibility, yet prognosis of allogenic BMT remains uncertain in most cases. In addition, immune system suppression is necessary, graft-versus-host disease (GVHD) issues are a concern, and the disease-free survival periods are still short. The availability of sibling donors and the quality of their transplants, such as due to age considerations, is always an issue too, reducing significance of this source of BMT.

Autological BMT, i.e., inoculation of the patient after supra-terminal cytotoxic treatment with his or her own bone marrow specimen that was collected after his or her diagnosis and usually soon before his supra-terminal cytotoxic treatment, presents no danger of GVHD or other immune system response. Since no suppression of the patient immune system is necessary, the short-tern survival rate after the transplantation is very high. Unfortunately, since the patient's bone marrow is collected during a post-diagnosis time frame when the disease has already spread extensively, conventional autological BMT runs the serious risk of re-introducing into the patient the very same malignant cells (s)he was just treated against via supra-terminal cytotoxic treatment. Various methods of purification, or “purging”, are currently being used and developed to rid harvested bone marrow specimens of malignant cells before reinfusion of the processed marrow back into the self-donor, but they all reduce the vitality of the transplant and they can never be 100% effective and selective. Moreover, reduction of malignant cell population in the transplant below the level of detection does not necessarily solve the re-introduction problem. It only secures some time until their population in the patient organism, weakened by the therapy, reaches the pre-treatment level again. And even with consecutive cytotoxic treatments, the restoration of the patient's hemopoiethic and immune systems is often accompanied by relapse of the malignancy. In addition, such purging processing unavoidably will damage some of the healthy cells in the marrow specimen. Therefore, a surplus of marrow beyond that otherwise needed for BMT therapy has had to be harvested from the ill patient to ensure enough marrow would remain available after purging to conduct the reinfusion-transplantation therapy. Also, the age and condition of self-donating patient has been shown to affect the ability of his or her bone marrow specimen to restart patient's hemopoiethic function.

Given these serious drawbacks and problems associated with prior BMT treatments, other alternate therapies may be considered. For instance, embryonic stem (ES) cells have been widely touted to represent a major potential for cell therapies for regenerative medicine. However, in addition to possible regulatory concerns and restrictions, this approach is considered less promising than touted since successful transplantation would still require induction of tolerance in recipients and ongoing immune suppression. The use of ES cells in transplantation may depend on the formation of a large bank of suitable human leucocyte antigen (HLA) types or the genetic erasure of their HLA expression. Although it is possible to customize ES cells by therapeutic cloning or cytoplasmic transfer, it appears very unlikely that these strategies will be used extensively for producing ES cells compatible for transplantation in foreseeable future.

References describing prior BMT and ancillary procedures include the following.

U.S. Pat. Nos. 5,913,859, 6,110,176 and 6,358,252 to Shapira describe an apparatus and method for extracting bone marrow from patients for subsequent collection and storage. More specifically, these patents describe a method and apparatus for obtaining bone marrow and bone marrow fluid from the jawbone of a patient with relative ease and minor discomfort before, during, or after dental procedures for long term storage and/or bone typing. These patents also describe the ability of an individual to collect and store his own bone marrow before the onset of any disease, such as childhood leukemias, which usually occurs between the ages of 15 and 30. The bone marrow is treated before storage or transplantation in an effort to protect a patient from a relapse caused by undetected cancer cells. However, the amount of bone marrow available to be extracted from a typical human jawbone is not considered to be adequate for supporting autological BMT-based treatments associated with the restoration of a patient's hemopoiethic and immune systems. Moreover, many dental patients can be expected to be suffering from serious dental and/or gum disease at the time the bone marrow would be orally extracted from their jaw bones when done at the time of a dental procedure that they are undergoing.

U.S. Pat. Nos. 5,004,681, 5,192,553 and 6,461,645 to Boyse et al. describe isolation and preservation of neonatal hematopoietic stem and progenitor cells of the blood derived from umbilical cord blood or placental blood of a single human collected at birth, cryopreservation of the collected cells, and thawing and using the stem cells in the treatment of diseases and disorders, including use for autologous reconstitution. The collection of stem cells from umbilical cord blood or placental blood in these patents is based on a premise that such cells will be present in an amount sufficient to effect hematopoietic reconstitution of a human adult.

U.S. Pat. No. 5,199,942 to Gillis describes methods for improving autologous hematopoietic cell transplantation in patients undergoing cytoreductive therapies, and particularly to methods in which bone marrow or peripheral blood progenitor cells are removed from a patient prior to myelosuppressive cytoreductive therapy, expanded in ex vivo culture in the presence of a growth factor, and then readministered to the patient concurrent with or following cytoreductive therapy to counteract the myelosuppressive effects of such therapy.

U.S. Pat. Nos. 5,759,764 and 5,580,714 to Polovina describe a cryopreservation solution in which cryopreserved and thawed umbilical cord cells, platelets, and hematopoietic stem and progenitor cells can be used therapeutically for reconstitution of the hematopoietic system in a suitable patient. The cells can be introduced by any method known in the art with systemic infusion generally preferred.

U.S. Pat. No. 6,277,557 to Burger, et al. describes an infusible grade short-term cell storage medium.

As can be appreciated from the above, a need remains, as well as opportunities exist, for innovative processing modalities in which a bone marrow transplant modality or other tissue restoration or rejuvenation therapy is provided that would cause no immune response problems and be very efficient in restoring patient's hematopoietic and immune systems and functions. The present invention meets these needs and fulfills these opportunities.

### SUMMARY OF THE INVENTION

This invention relates to a method for treating a disease, disorder, or serious medical condition in a mammal, including the steps of harvesting a biological specimen containing stem cells from the body of a donor, and then storing the harvested stem cell specimen for an appropriate waiting period until after the donor contracts and is diagnosed with one or more of a cytopthalogical illness, a chronic fatigue syndrome, and/or damaged tissue. In one preferred embodiment, the stem cells are harvested from the peripheral blood of the donor. In another embodiment, the stem cells are harvested from the donor's bone marrow. When harvested from the peripheral blood, preferably, the donor is pretreated with a stem cell growth stimulating agent in a manner effective to stimulate increased presence of stem cells in the peripheral blood prior to donation. After the donor subsequently contracts and is diagnosed with a cytopthalogical disease, damaged or deteriorated tissue, and/or a chronic fatigue syndrome, at least a portion of the stored biological specimen that contains stem cells is reintroduced in therapeutic amount in the donor, where the reintroduction occurs either after any cytotoxic therapy is performed on the donor that damages native bone marrow, or as part of another medical treatment used to reconstitute or restore healthy tissue, in the donor.

In one preferred embodiment of the present invention, stem cells are collected from the peripheral blood of a person, or other type of mammal, the donor, who need not be prescreened as being free of malignant disease by clinical and laboratory testing methods before cryopreservation of the bone marrow specimen harvested from the donor, as the passage of a predetermined period of time during storage of the harvested specimen will effectively reveal whether the donor had a cancer or other malignancy at the earlier time of donation. If the donor does not develop a cancer within the predetermined waiting period, as a pseudo-quarantine period, then the donated stem cell need not be purged via conventional processing methodologies to remove tumor cells or otherwise decontaminate it before its re-infusion back into the donor when needed by that donor at a later date for for cell reconstitution as an adjunct treatment of a malignant disease, disorder or malady that the donor has acquired after donation. For instance, after primary treatment of a cancer using high-dose chemotherapy or the like to destroy cancer cells, autologous transplantation of the patient's own stem cells, which have not been deterioriated by standard purging-decontamination processing, according to an embodiment of the present invention makes it possible for the body to regenerate its ability to produce blood cells, including immune system cells needed to fend off disease. By harvesting stem cell samples from donors before they are diagnosed with a cancer or other malignancy and then subjecting the donated sample to a predetermined waiting period to confirm the donor's “cancer- or disease-free good health” at the earlier time of donation, then the need to perform purging procedures or other special processing procedures that diminish the quality and quantity of harvested sample is avoided in the practice of the present invention.

It will be appreciated that the “predetermined waiting period” used a screening method is not necessarily an invariable fixed period of time, but can vary depending on the circumstances. As generally known, different types of malignant cancers can develop and grow at different rates, and the development and growth rate can vary from person to person. As a general rule of thumb, however, the longer the waiting or “quarantine” period, the less likely the donor was suffering unnoticed from a malignant disease at the time of donation and that the donated specimen containing stem cells was likely to be contaminated with malignant cells. In one embodiment, the predetermined waiting period for storing the donated specimen after harvesting it until re-infusion back into the same donor ranges from about 12 months or more, and preferably is at least about 60 months or more. A specimen stored for several years, especially five years or more, will be sufficient to protect against specimen contamination from the vast majority of all malignant cancers of greatest concern which can be expected to have a noticeable onset in patients well within this time frame. Again, an objective of the invention is to provide a screening methodology that makes it possible to avoid the use of purging processes on, and their resulting damage to, donated samples of stem cells before its re-infusion.

For purposes herein, “stem cells” mean cells that have the ability to divide for indefinite periods in culture and to give rise to specialized cells. This definition encompasses, but is not limited to, progenitor cells, i.e., “committed” stem cells. In one preferred embodiment, the stem cells are hematopietic stem cells, and in a further preferred embodiment are adult hematopietic stem cells and not prenatal or neonatal hematopietic stem cells. In the present invention, stem cells can be harvested from mature children or older donors at the convenience of the donors while reducing and avoiding the added medical costs and degradation in therapeutic potency associated with standard purging procedures conventionally used on specimens containing adult stem cells as harvested only after diagnosis with a malignante disease.

### BRIEF DESCRIPTION OF THE DRAWINGS

Other features, objects, and advantages of the present invention will become apparent from the following detail description of preferred embodiments of the invention with reference to the drawings, in which:

FIG. 1 is a flow chart of a prior art method for a cancer therapy using BMT.

FIG. 2 is a flow chart of another prior art method for a medical therapy using BMT.

FIG. 3 is a flow chart of a method for a medical therapy using autologous stem cell transplantation according to an embodiment of the present invention.

### DETAILED DESCRIPTION OF THE PREFERRED EMBODIMENTS

Stem cells are harvested as a specimen obtained from the peripheral blood or bone marrow of a donor for cryopreservation and storage and held for a predetermined waiting period until a later date for autologous transplantation back into the donor when useful for cell restoration and/or rehabilitation as a primary treatment for a condition of damaged, deteriorated, and/or fatigued tissue in the donor, for which the donor has contracted after the time of donation. In this manner, the necessity and associate cellular damage associated with conventional purging techniques that otherwise must used on stem cell specimens drawn only after diagnosis of a malignant disease in the donor is averted. Therefore, a more robust and viable sample of the donor's stem cells are available for re-infusion as part of a tissue restoration or rejuvenation therapy being performed on the donor, and consequently the donor's outcome to the therapy can be improved and enhanced.

Referring now to FIG. 3, a medical therapy according to an embodiment of the present invention is shown that relates to treatment of cytopathological disease or other medical conditions in a mammal using autologous transplantation techniques involving: choosing between harvesting stem cells from the peripheral blood or another source of stem cells in a prospective donor, such as bone marrow (301), administering a stem cell growth stimulating agent if peripheral blood is chosen as the source of stem cells from the donor (302), and if not, then proceed directly to the next step of harvesting the stem cells from the donor; harvesting a biological specimen containing stem cells selected the group consisting of peripheral and bone marrow from the body of the healthy donor (303); cryopreserving and storing the harvested biological specimen of stem cells (304); and after (and, implicitly, if) the donor is later diagnosed with a cytopthalogical disease, damaged or deteriorated tissue, and/or a chronic fatigue syndrome, then a check is made to determine how long the specimen has been in storage (305), and if the specimen meets a predetemined storage period applicable to that donor and a cancer or cancers of concern, then the treatment can proceed on directly to therapy in steps 306/308 or 310, and, if not, the speciment must be purged or otherwise decontaminated before it can be used in a therapy regimen; then, reintroducing at least a portion of the stored biological specimen that contains stem cells in therapeutic amount in the donor after the donor is diagnosed with a condition for which stem cell re-infusion therapy is appropriate or otherwise applicable (308/309 or 310). In the instance of a therapy calling for administering a high dose of chemotherapy to the donor (308), the reintroduction occurs after that or other cytotoxic therapy is performed on the donor that damages native bone marrow (308). As shown by step 307, if the colelcted stem cell has not been stored for a period of time sufficient to meet the prescribed waiting period, then it must be purged before re-infusion in the donor to avoid the use of a sample potentially contaminated with tumor cells and the like.

Regarding step 305 in more detail, the longer the waiting or “quarantine” period, the less likely the donor was suffering unnoticed from a malignant disease at the time of donation and that the donated specimen containing stem cells was likely to be contaminated with malignant cells. In one embodiment, the predetermined waiting period for storing the donated specimen after harvesting it until re-infusion back into the same donor ranges from about 12 months or more, and preferably is about 60 months or more.

Regarding step 302 in more detail, a stem cell growth stimulating agent or mobilizing dose of chemotherapy is administered to the donor to increase stem cell levels in the donor. The stem cell growth stimulating agent can be agents generally known to have this affect, such as granulocyte-colony stimulating factor or other chemotherapeutic agents. The prospective donor is administered the stem cell growth stimulating agent in advance of collection of an apheresis specimen from the donor. For example, granulocyte-colony stimulating factor can be administered to the donor in an amount of 3-15 g/kg/day for 1-10 days before collection.

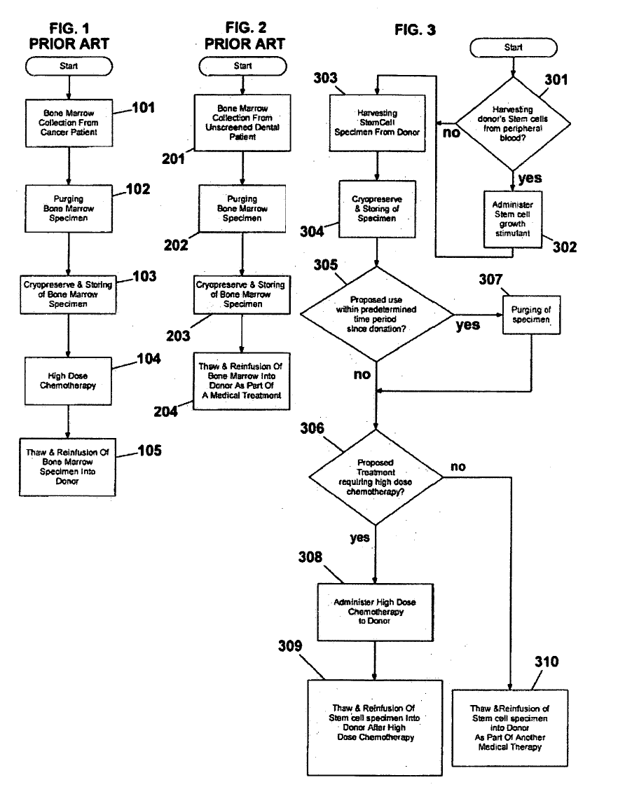

By comparison, some prior art methodologies, such as those illustrated in FIG. 1 (generally summarized as steps 101, 102, 103, 104, 105) and FIG. 2 (generally summarized as steps 201, 202, 203, 204), do not medically screen out unsuitable donor candidates before proceeding to harvest bone marrow from a donor. In addition, the prior art methods illustrated in FIGS. 1 and 2 include a purging operation used to remove malignant tumor cells from the harvested bone marrow. The present invention makes it possible to avoid such processing of the harvested stem cells that would reduce the quality and yield of the healthy tissues and cells in the harvested stem cell sample.

It optionally is possible, although not required, in the present invention to subject the donor and/or the donated specimen, before re-infusion of the donated specimen, to blood screening or immunoassay tests and the like for detection of cancer markers. These tests generally are not useful to test for all types of cancer or even great numbers of types of cancers, and/or they are not 100% reliable and accurate, as the level of detection can vary greatly. If there is a clear indication of the type of cancer involved, a laboratory cancer test may be selected and applied with more efficacy in cases. If such optional laboratory screening tests are applied in the practice of the invention, they are merely used to supplement the use of the waiting period during cryopreserved storage as the primary screening mechanism.

In any event, the screening tests that optionally can be included as a secondary screening measure, include assays effective for presymptomatically detecting and diagnosing cancers. Techniques are generally known for identifying cancer-specific serum protein markers via a screening strategy. E.g., see Watkins, et al., “Detection of early-stage cancer by serum protein analysis,” americanlaboratory, 32-36, June 2001. For instance, as explained by Watkins et al., known and available biomarker screening tests include urinary NMP22® for bladder cancer and serum prostate-specific antigen (PSA) testing for prostate cancer. Fecal occult blood testing is available for colon cancer detection, mammography for breast cancer, alpha-fetoprotein for heptacellular carcinoma and testicular cancer, catecholamines for neuroblastomo, and immunoglobulins for multiple myeloma, and so forth. Also, proteomic analysis by surface-enhanced laser desorption/ionization (SELDI) is an improved method enabling detection of cancer-specific proteins in complex biological mixtures such as serum after processing to remove major classes of interfering serum components. The above-mentioned screening tests are illustrative and not exhaustive in nature. Persons knowledgeable in this field of endeavor will appreciate other screening strategies that can be applied as needed or useful in screening potential autologous BMT or PBSCT donors.

In a preferred embodiment, the donor's own blood or bone marrow is used as the source of stem cells, and to be potent and lively it has to be collected when the patient is still determined to be healthy. In a preferred embodiment, the stem cell specimen is harvested while the donor is postnatal, such as from a young child, teenager or adult. The harvesting of the stem cell sample can be performed on an out-patient basis, especially in the case of stem cell collection via apheresis. If bone marrow is instead used as the source of the stem cells, it can collected from during a normal routine medical procedure performed on many healthy children or teenagers while the donor is under general anesthesia, such as during a tonsillectomy or adenoidectomy. Also, many otherwise healthy youths undergo a wide variety of surgical procedures to correct ophthalmic conditions or other congenital defects, which require general anesthesia, and thus provide excellent opportunities to collect healthy bone marrow from the healthy youth.

Of course, there is no requirement that a donor wait until he or she undergoes an unrelated surgical procedure to extract the bone marrow. For example, to the extent a donor's family medical history indicates a person is at high risk for potentially needing stem cell infusion therapies or BMT in the future should they contract a malignancy, the donor may deem it advisable to have his or her stem cell specimen harvested at an early date while the donor is still healthy without waiting until it can be done during an unrelated surgical procedure.

The harvesting of the stem cell sample for future autologous transplantation can be accomplished by any customary or suitable medical procedure used for that purpose. If stem cells are sourced from peripheral blood of the donor for purposes of supporting a peripheral blood stem cell transplantation (PBSCT) modality of the invention, the removal of stem cells from the circulating blood can be accomplished by machine as part of a customary apheresis procedure. In the case of bone marrow extraction, the collection can be performed such as by making one or more insertions of a syringe means into the donor's hip or pelvic bone to extract bone marrow as the biological specimen containing stem cells. Cryogenic preservation generally will be the preservation technique of choice for the harvested biological specimen that contains stem cells. Cryogenic preservation allows for short-term or long-term time storage so that the healthy stem cells will be available in revitalized condition if and when a disease strikes. In general, cryopreservation involves combining the harvested stem cell specimen with a suitable preservative, such as dimethyl sulfoxide (DMSO), before storage in a liquid nitrogen freezer. The stored specimen is removed from the freezer and thawed before use in a medical procedure. Cryopreservation techniques are generally known that can be applied and adapted to this invention to provide long-term cryopreservation of a donor's harvested peripheral blood, or bone marrow, containing stem cells. In this regard, reference is made, for example, to U.S. Pat. Nos. 5,192,553; 5,580,714; and 6,277,557, and the references cited in the relevant part in each of these references, which teachings are incorporated herein by reference.

Suitable public and private cryopreservation depositories, institutions and facilities exist and can be used for arranging the storage of a donor's harvested stem cell speciment until needed at a future date after a medical need therefor arises in the donor. The present invention, in one embodiment, also is envisioned as being implemented via a nation-wide service for collection, cryogenic preservation, storage and rehabilitation of personal stem cell specimens from young and/or otherwise healthy children or adults willing to provide the best possible material for their own autologous transplantation therapy in case they ever need it.

The medical conditions and diseases to which the application of autologous transplantation according to the treatment regimen of the present invention is is not necessarily limited. For instance, as one category of treatments, the present invention is applicable to the treatment of a cytopathological disease in a mammal. For example, the stem cell speciment collected from the healthy donor, cryopreserved, and stored for the waiting period, can be therapeutically reintroduced back into the same donor when needed in the future to treat illnesses and disorders such as bone marrow damage caused as a result of “supra-terminal” cytotoxic treatments of cancer that has formed in the patient after his or her earlier stem cell donation. For example, autologous BMT is used in a therapy to reconstitute and rescue damaged and destroyed bone marrow resulting from supra-terminal cytotoxic treatments, such as high dose chemotherapy or radiotherapy treatments, and to restore normal hematopoietic and immunlogical function in the donor after such cytotoxic treatments. In one embodiment, stem cell transplants via BMT can be used in the treatment of cancers and leukemias. The major indications are hematological malignancies such as acute myeloid leukemia (AML), acute lymphoblastic leukemia (ALL), chronic myeloid leukemia (CML) and myelodysplastic syndromes, and in the instance of solid tumors, breast-, testicular-, small cell lung-, and/or ovarian cancers.

The treatment regimen of the present invention also can be applied to stem cell or BMT therapies for infectious diseases, viral and otherwise, that attack and destroy blood and/or bone marrow cells (e.g., HIV), and so forth. The treatment regimen additionally could be applied to stem cell transplantation, such as BMT therapies, for terminal radiation or chemical overexposure (poisoning) that can be treated by BMT, including military, space exploration, and high risk emergency and rescue team personnel.

The reintroduction of the stored stem cell speciment also can be used as part of an autologous stem cell transplantation, such as BMT procedure, to provide tissue and/or organ restoration by inoculating said tissue and/or organ with specially conditioned stem cells present in the harvested biological specimen that contains stem cells. Other examples of illnesses and disorders that can be treated according to the invention include, for example, neurological disorders, such as Alzheimer's disease or Parkinson's disease; a liver disease; diabetes; a thyroid gland disorder; anemias, and so forth. For instance, liver tissue destroyed by trauma or deterioration, e.g., cirrhosis, damage from viral hepatitis, and so forth, can be treated according to this invention. Pancreatic tissue also can be treated as a therapy for a diabetic condition that has emerged in the donor after donation.

As another general category of therapy, the present invention can be generally applied to restoring damaged or deteriorated condition that has arisen in the donor since donation, such as an anatomical system of the mammal selected from the group consisting of damaged or deteriorated hematological system tissues, damaged or deteriorated immune system tissues, damaged or deteriorated muscular system tissues, damaged or deteriorated neurological system tissues, a damaged or deteriorated cardiovascular system tissues, damaged or deteriorated renal system tissues, damaged or deteriorated lymphatic system tissues, damaged or deteriorated liver tissues, deteriorated dermatological system tissues, damaged or deteriorated reproductive system tissues, individually or in combination. For instance, the invention could be applied to treating damaged or deteriorated reproductive system tissues, such as testicular tissues, in order to treat male infertility.

As yet another general category of therapy, the present invention also can be generally applied to rejuvenating a mammal that is chronically fatigued or as part of an anti-aging therapy. For instance, the method of the invention could be applied to treat a fatigued condition that has arisen in the donor involving a fatigued hematopoietic system, a fatigued immune system, a fatigued muscular system, a fatigued neurological system, a fatigued lymphatic system, individually or in combination. The invention also can be applied for general stimulation of hematopoietic and immune systems in a patients or as a general anti-aging therapy.

As can be appreciated, this invention addresses an area of broad potential area of medical application where healthy and immunocompatible stem cells can be a powerful instrument of restoration of a normally functioning tissue, such as in the treatment of cell-degenerative diseases, such as diabetes, Parkinson's disease, liver cirrhosis, and so forth. In addition, it was previously thought that cells within bone marrow solely functioned to regenerate cells within the marrow, as well as all circulating hematopoietic cells in peripheral blood. Recent reports, however, suggest that marrow-derived cells can also regenerate other cell types, including cardiac muscle, liver cell types, neuronal and non-neuronal cell types of the brain, as well as endothelial cells and osteoblasts.

Stem cells are the natural units of embryonic generation, and also adult regeneration, of a variety of tissues. It is possible that all organs and tissues are derived from, and still contain, stem cells. Because the number and activities of stem cells and their progeny are homeostatically regulated, it is thought that the present invention greatly bolsters clinical stem cell transplantation capabilities to greatly add to the physician's armamentarium against degenerative diseases and other medical conditions. The present invention helps to harness the potential of adult stem cells for autologous cell and gene therapy. The recent discovery of stem cells in the mature human brain was a remarkable finding given that neurological tissue was previously believed incapable of generating new neurons; but neuropoiesis is now an established phenomenon in the adult brains of mammals, including human beings. This persistent neurogenesis has potential therapeutic applications for various neurological disorders as a source for tissue engraftment and as self-repair by a person's own indigenous population of previously collected and preserved pluripotent stem cells from bone marrow or biogenic by-products of their proliferation and differentiation. The present invention can be used to implement such emerging therapeutic applications.

In addition, one of the many proposed applications for anti-cancer therapy is the transfer of drug-resistance genes into bone marrow stem cells for myeloprotection. Protection of the hosts' bone marrow in this particular case should allow for dose escalation that may be useful for eradicating minimal residual disease in a post-transplant situation. There are a number of drug resistance genes, whose products include mutant forms of enzymes that confer resistance to chemotherapeutic drugs. Advances in hematopoietic stem cell isolation and ex vivo manipulation have kept pace with improvements in retroviral vector technology to make hematopoietic stem cell transduction a distinct reality. Clinical trials, which have established that the approach is safe, are now being designed to address more therapeutically relevant issues. Again, the present invention can be used to implement such emerging therapeutic applications.

While the invention has been described in terms of preferred embodiments, those skilled in the art will recognize that the invention can be practiced with modification within the spirit and scope of the appended claims.

What is claimed is:

1. Method for treating a cytopathological disease in a mammal, comprising:
(a) harvesting a biological specimen containing stem cells from the body of a donor;
(b) storing at least a part of said biological specimen that contains stem cells for a predetermined period time without being re-infused back into the donor;
(c) reintroducing at least a portion of said stored part of the biological specimen containing stem cells in therapeutic amount in the donor after the predetermined period of time and after said donor is diagnosed with a cytopthalogical illness or damaged tissue in need of rejuvenation, wherein said re-introduced specimen is not purged prior to re-infusion.

2. The method according to claim 1, wherein said biological specimen containing stem cells comprises peripheral blood.

3. The method according to claim 1, wherein said stem cells comprise adult hematopietic stem cells.

4. The method according to claim 1, further including a step of introducing a stem cell growth stimulating agent into the donor prior to the harvesting in a manner effective to increase the population of the stem cells in the peripheral blood of the donor before harvesting the specimen.

5. The method according to claim 1, wherein said biological specimen containing stem cells comprises bone marrow.

6. The method according to claim 1, wherein said cytopthalogical illness is cancer, and further including a step between (b) and (c) of subjecting said donor to a treatment regimen selected from the group consisting of chemotherapy or radiotherapy.

7. The method according to claim 1, wherein said cytopthalogical illness comprises an infectious disease that attacks and damages blood and/or bone marrow cells.

8. The method according to claim 1, wherein said cytopthalogical illness comprises HIV.

9. The method according to claim 1, wherein said cytopthalogical illness comprises terminal radiation or chemical overexposure poisoning.

10. The method according to claim 1, wherein said reintroducing step comprises providing tissue and/or organ restoration by inoculating said tissue and/or organ with specially conditioned stem cells present in the harvested biological specimen containing stem cells.

11. The method according to claim 1, wherein said cytopthalogical illness comprises a neurological disorder.

12. The method according to claim 1, wherein said neurological disorder is selected from the group consisting of Alzheimer's disease and Parkinson's disease.

13. The method according to claim 1, wherein said cytopthalogical illness comprises a liver disease.

14. The method according to claim 1, wherein said cytopthalogical illness comprises diabetes.

15. The method according to claim 1, wherein said cytopthalogical illness comprises a thyroid gland disorder.

16. The method according to claim 1, wherein said harvesting comprises making one or more insertions of a syringe means into the donor's hip or pelvic bone to extract bone marrow as said biological specimen containing stem cells.

17. The method according to claim 1, wherein said storing comprises cryopreservation.

18. The method according to claim 1, wherein said mammal comprises a human.

19. Method for restoring damaged and/or deteriorated tissue in a mammal, comprising:
(a) harvesting a biological specimen containing stems cells from peripheral blood of the body of a donor;
(b) cryopreserving and storing at least a part of said biological specimen that contains stem cells for a predetermined waiting period without being re-infused back into the donor;
(c) reintroducing at least a portion of said stored part of biological specimen that contains stem cells in therapeutic amount in the donor after the donor the predetermined period of time and after said donor has been diagnosed as having a damaged and/or deteriorated tissue condition, wherein said re-introduced specimen is not purged prior to the re-infusion.

20. The method according to claim 19, wherein said stem cells comprise adult hematopietic stem cells.

21. The method according to claim 19, further including a step of introducing a stem cell growth stimulating agent into the donor prior to the harvesting in a manner effective to increase the population of the stem cells in the peripheral blood of the donor before harvesting the specimen.

22. The method according to claim 19, wherein said damaged or deteriorated condition comprises an anatomical system of the mammal selected from the group consisting of damaged or deteriorated hematological system tissues, damaged or deteriorated immune system tissues, damaged or deteriorated muscular system tissues, damaged or deteriorated neurological system tissues, a damaged or deteriorated cardiovascular system tissues, damaged or deteriorated renal system tissues, damaged or deteriorated lymphatic system tissues, damaged or deteriorated liver tissues, deteriorated dermatological system tissues, damaged or deteriorated reproductive system tissues, individually or in combination.

23. The method according to claim 19, wherein the predetermined waiting period is at least 12 months.

24. The method according to claim 19, wherein the predetermined waiting period is at least about 60 months.

25. The method according to claim 19, wherein said mammal comprises a human.

26. Method for rejuvenating a chronically fatigued mammal, comprising:
(a) harvesting a biological specimen containing stems cells from peripheral blood of the body of a donor;
(b) cryopreserving at least a part of said biological specimen that contains stem cells for a predetermined waiting period without being re-infused back into the donor;
(c) reintroducing at least a portion of said stored part of biological specimen that contains stem cells in therapeutic amount in the donor after the predetermined period of time and after said donor has been diagnosed has having a chronic fatigue condition, wherein said re-introduced specimen is not purged prior to the re-infusion.

27. The method according to claim 26 wherein said stem cells comprise adult hematopietic stem cells.

28. The method according to claim 26, further including a step of introducing a stem cell growth stimulating agent into the donor prior to the harvesting in a manner effective to increase the population of the stem cells in the peripheral blood of the donor before harvesting the specimen.

29. The method according to claim 26, wherein said fatigued condition comprises an anatomical system of the mammal selected from the group consisting of a fatigued hematopoietic system, a fatigued immune system, a fatigued muscular system, a fatigued neurological system, a fatigued lymphatic system, individually or in combination.

30. The method according to claim 26, wherein said mammal comprises a human.

31. The method according to claim 26, wherein the predetermined waiting period is at least 12 months.

32. The method according to claim 26, wherein the predetermined waiting period is at least about 60 months.
